(19) United States
(12) Patent Application Publication
(10) Pub. No.: US 2002/0193522 A1
(43) Pub. Date: Dec. 19, 2002

(54) Synthesis and luminescent characteristics of novel phosphorus containing light-emitting polymers

(76) Inventor: Yih-Min Sun, Tainan Hsien (TW)

Correspondence Address: BACON & THOMAS, PLLC, 625 SLATERS LANE, FOURTH FLOOR, ALEXANDRIA, VA, 22314

(21) Appl. No.: 09/879,963

(22) Filed: Jun. 14, 2001

(51) Int. Cl. C08F 8/00

(52) U.S. Cl. 525/168

(57) ABSTRACT

The present invention relates to a kind of synthesis and luminescent characteristic of novel phosphorus containing light-emitting polymers, especially one improving the luminescence efficiency of the synthesis light-emitting polymers. According to the method of the present invention, the electron-transporting chromophores are introduced into an emission polymer to increase its electron affinity. Further, several phosphorus-containing emission chromophores are synthesized and incorporated with electron-transporting chromophores finally resulting in the novel phosphorus chromophores emitting blue light as expected, improving thermal stability of resulting polymers such that the absorption peaks of these polymers are restricted to a stable range.

### TECHNICAL RANGE

The present invention relates to a kind of synthesis and luminescent characteristic of novel phosphorus containing light-emitting polymers, especially one improving the illuminating efficiency of the synthesis light-emitting polymers.

### INVENTION BACKGROUND

Light-emitting diodes (LEDs) are extremely important optoelectronic materials and have been applied in a variety of products such as electronic and optoelectric commercial products. Although the flat panel displays that utilize electroluminescent technology have already been on the market since 1997, they are all made of small molecule. For example, Pioneer Company's green electroluminescent has used quinacridone. However, for better luminescence efficiency or intensity, all the fluorescence dyes of small molecule have to be mixed with other materials. The mixed materials have such bad compatibility that they bring about detachment between the mixed materials. Furthermore, the process of using vapor deposition procedure to produce light emitting-devices is quite complicated and it is very difficult to make panels with large area using this process. Since the discovery by Holmes and others in the U.K of the electroluminescent characteristics, many polymers that can be used to fabricate LEDs have been synthesized to compensate for the drawbacks of liquid crystals which pose difficulties in the fabrication of large-area display panels.

In the 1990s, Holmes and others first discovered electroluminescent materials by means of poly (1,4-phenylene vinylene) (PPV) which can emit green-yellow light (peak wavelength between 520 and 551 nm). PPV has the advantages of being lightweight, having large size, flexibility and ease of fabrication into flat panel displays, which are the general properties of polymeric materials. PPV's tensile strength is better than that of polyaramide and can survive severe conditions during device processing.

The electroluminescence (EL) devices using organic dyes are made by vacuum technique, which lines up the molecule very regularly in films and result in lesser number of traps in the films. Thus, they have high luminescence efficiency. They are, however, not stable and have weak mechanical strength. The molecule of a polymer is very difficult to purify and to line up regularly and also contains lots of traps. So the electrons and holes are easy to be captured by traps, and thus will result in a loss of energy.

### SUMMARY OF INVENTION

The present invention relates to a kind of synthesis and luminescent characteristic of novel phosphorus containing light-emitting polymers, incorporating novel phosphorus chromophores and electron-transporting chromophores and synthesizing several phosphorus-containing emission chromophores and luminescent polymers.

The present invention relates to a kind of synthesis and luminescent characteristic of novel phosphorus containing light-emitting polymers, and with the advantages of the emission chromophores of the polymers, we can improve the drawbacks of the conjugated polymers that show photoluminescence in the long wavelength range.

The present invention relates to a kind of synthesis and luminescent characteristic of novel phosphorus containing light-emitting polymers, using 2,5-bis-(-fluorophenyl)-1,3,4-oxadiazole incorporated with three derivatives of DOPO to provide nucleophilic substitution reaction of oxadiazole-activated bis(halide) monomers with his (phenol) monomers resulting in aromatic polyethers.

### DESCRIPTION OF DRAWINGS

FIG. 1 shows the synthesis of BFO.

FIG. 2 shows the synthesis of derivatives DOPO, DOPO-BQ, DOPOP-BQ, DOPO-NBP.

FIG. 3 shows the 1,3,4-oxadiazole activating the ortho- and para-substitution group in this reaction.

FIG. 4 shows the photoluminescence spectra of the DOPO derivatives.

FIG. 5 shows the synthesis of three kinds of polymers P1, P2 and P3.

FIG. 6 shows the testing of thermal stability.

FIG. 7 shows the Vis absorption of the polymers in solution.

FIG. 8 shows the Vis absorption of the polymers in thin films.

FIG. 9 shows the photoluminescence spectra of the polymers.

FIG. 10 shows the table of maximum absorption and photoluminescent in film or solution.

FIG. 11 shows the table of maximum UV-Vis absorptions of polymers in different polar solvents.

### MODE FOR CARRYING OUT INVENTION

First step: Before use, all reagents and solvents are reagent grade or purified by standard methods and each spectrum, melting points, nuclear magnetic resonance and optical characteristics are analyzed elementally.

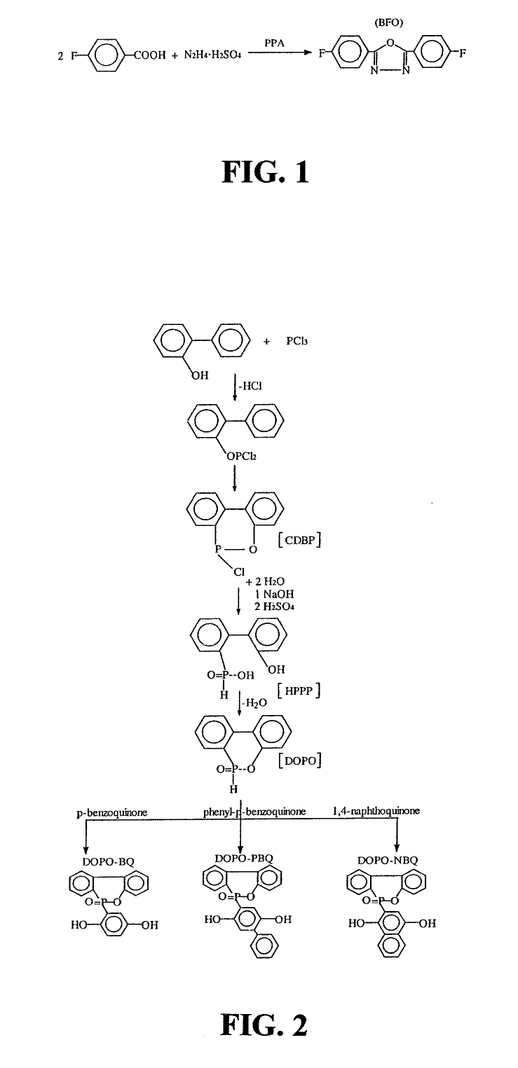

Second step: 2,5-bis(4-fluorobenzoic acid)-1,3,4-oxadiazole is synthesized; to a reaction vessel is added (N₂H₄.H₂SO₄) 5 mmol and (4-fluorophenyl) 10 mmol, and 30 g polyphosphoric acid (PPA) solvent. The mixture is first heated to 150° C. for 8 h. to dissolve the reactants and then the reaction mixture is further heated at cyclization temperature (200° C.) for another 2 h. After completion of the reaction, the mixture is poured into deionized water to a white color precipitate. The precipitate is recrystallized from ethanol and dried to obtain a transparent gray crystal. Yield 73.9%; m.p 203˜204° C. This synthesis procedure is shown in FIG. 1.

Third step: synthesis of DOPO [2-(6-oxido-6H-dibenz<c,e><1,2>Oxaphosphorin-6-yl)] derivatives and [DOPO-BQ(p-benzoquinone), DOPO-PBQ(phenyl-p-benzoquinone), DOPO-NBQ (1,4-naphthoquinone)]. Derivatives of DOPO are synthesized according to FIG. 2 and are further recrystallized from ethoxyethanol.

Fourth step: synthesis of organic light-emitting polymer containing phosphorus. 1 mmol of 2,5-Bis(4-fluorophenyl)-1,3,4-oxadiazole, 1 mmol DOPO derivatives and 2.1 mmol K₂CO₃, 10 ml toluene and 5 ml NMP/CHP (N-methyl-2-pyrrolidone/N-cyclohexyl-2-pyrrolidone vol. ratio 1:1). The mixture is heated to 150° C. for 2 h under nitrogen and toluene is collected by a Dean-Stark trap. Then excess xylene is removed and the reaction mixture is heated at reflux (180° C.) for 20 h. After reaction, the mixture is poured into 300 ml acetone/methanol (vol. ratio 1:1), diluted with addition to 5 ml NMP and further stirred overnight. The precipitate is further filtered, washed with distilled water, extracted with chloroform for 24 h and dried under vacuum. The products are thus finally obtained.

### Resuls

The present invention mainly synthesizes polymers containing electron-transporting chromophores in the main chain. The emission wavelengths of these synthesized polymers are adjusted by varying the molecular structures. The synthetic strategy of the desired monomers from simple staring materials is outlined in FIG. 1 and FIG. 2. The structures of these products are characterized by IR-, NMR spectroscopies, elemental analysis and by DSC and TGA to analyze the thermal properties of these polymers. The optical properties including absorption and luminescence of these polymers are measured with UV-Vis and PL systems.

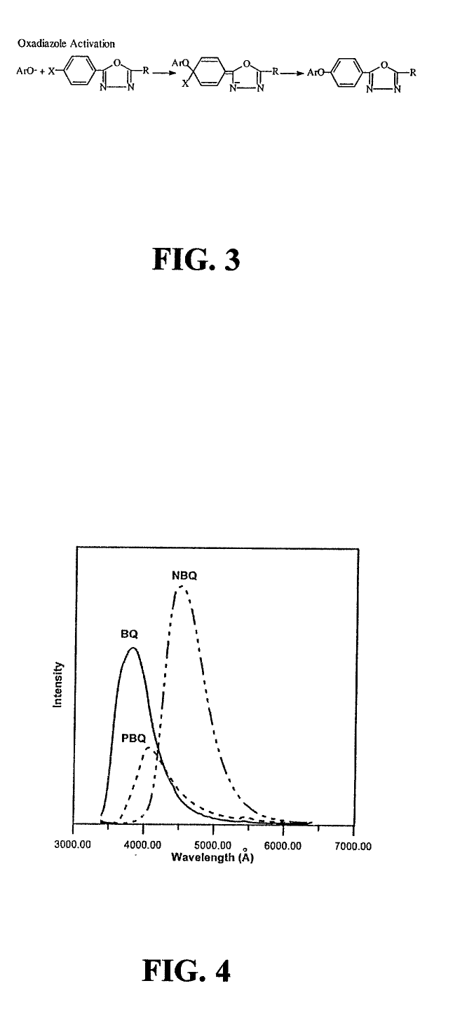

In this present case, two kinds of monomers are synthesized. 2,5-Bis(4-fluorophenyl)-1,3,4-oxadiazole (BFO) is the electron-transporting chromophore in polymers. Since 1,3,4-oxadiazole is a heterocyclic compound with less electrons, it can increase the electron affinity of the polymers. The other advantage is that it can disperse negative charges in the transition state when aromatic polyethers are synthesized, so the transition state will be stable and the reaction can be carried out more easily. As shown in FIG. 3, the ketone group activates X in this reaction, the 1,3,4-oxadiazole can also activate the ortho- and para-substitution group in this reaction.

In this present case, the DOPO derivatives are used as emission chromophores in the polymer. The DOPO derivatives include benzene, biphenyl or naphthalene ring structure as luminescent units. The PL spectra of the DOPO derivatives are shown in FIG. 4. The peak luminescent wavelengths are 388, 405 and 450 nm for DOPO-BQ, DOPO-PBQ and DOPO-NBQ respectively. It is apparent that the naphthalene ring has shifted the wavelength more towards the red than biphenyl ring because of its conjugated nature. The results are in agreement with our expectation that the spectra of DOPO-PBQ and DOPO-NBQ should emit light in the blue emission region. Therefore, the DOPO series can be adapted as precursors for organic light-emitting materials.

### Proof

When the polymer LED devices operate at some voltage, the temperature of the device will increase. So the thermal stability of polymers is very important. Tg of polymers must be as high as possible to avoid the loss of mechanical strength when the operating temperature is high. If the polymer crystallizes, it may be separated in phases, thereby reducing the device efficiency.

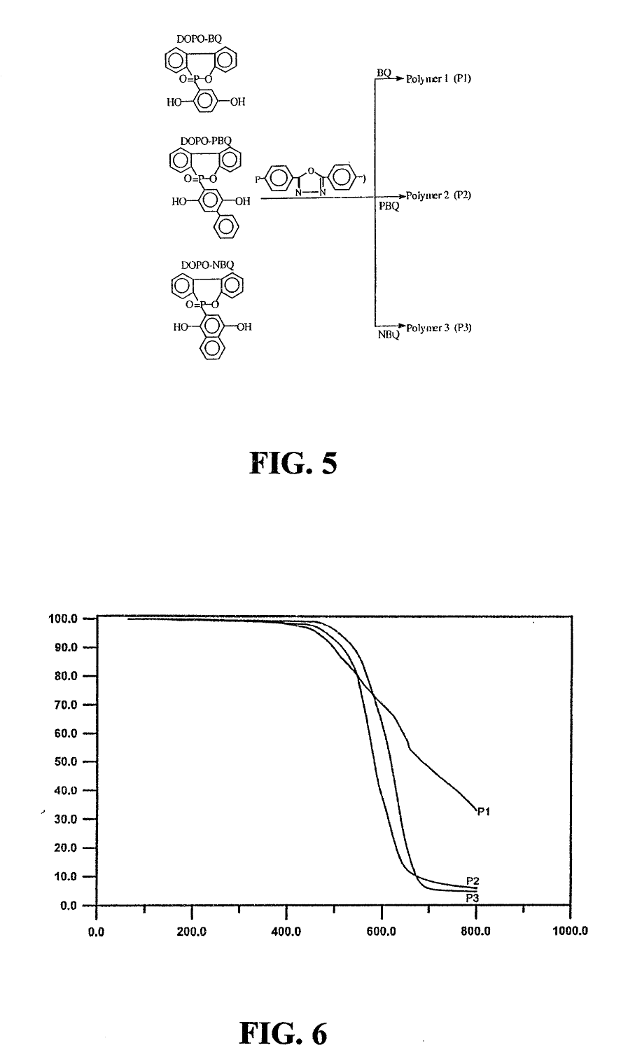

In order to understand the physical and optical properties of the synthesized polymers, three kinds of polymers, P1, P2 and P3 are synthesized (FIG. 5). The effects of the extended rings, benzene, biphenyl and naphthalene on the luminescent properties are studied.

From the results of TGA as shown in FIG. 6, it is apparent that these polymers have excellent thermal stability. The starting temperatures of degradation are all higher than 480° C. for P1, P2 and P3, thus showing high thermal resistance of 1,3,4-oxadiazole. The drastic decrease in weight is observed when the temperature is close to 480° C. which may be due to the degradation of the DOPO chain.

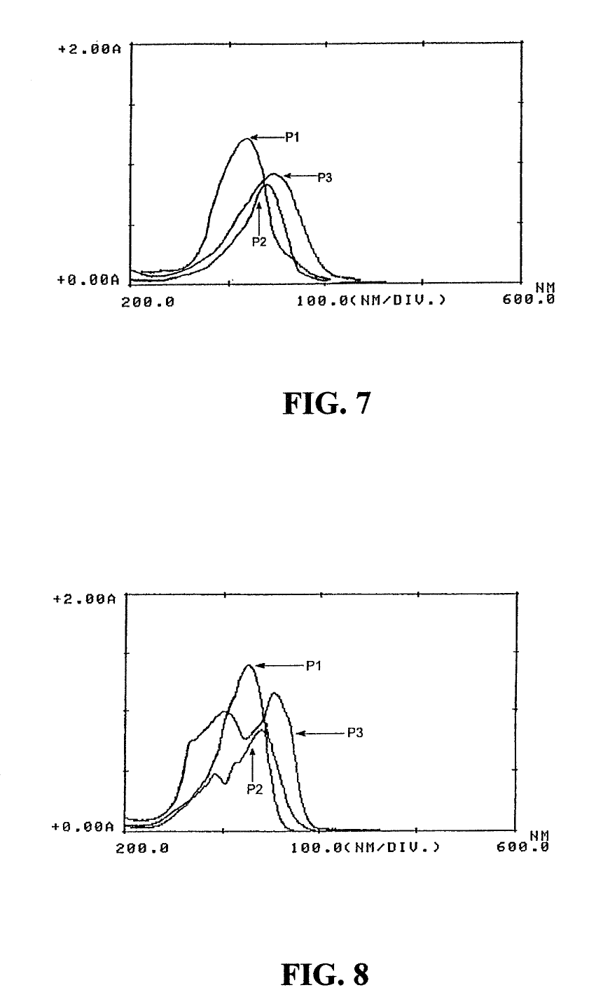

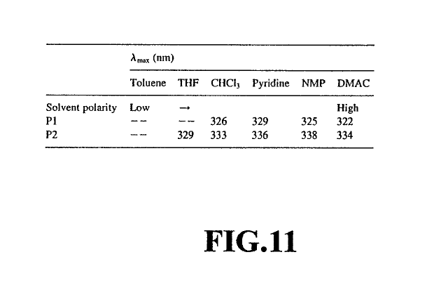

The emitting wavelengths of the EL polymers depend on the structure of the polymers. The absorption and PL spectra are measured. FIGS. 7 and 8 show the UV-Vis absorption spectra of the polymers in solution and as thin films, respectively. The peak absorption wavelengths are shown in FIG. 10. From FIG. 10, it is found that the polymer solution has apparently shifted the wavelength more to blue than thin film type which may be attributed to the expansion of polymer chain in the solvent. The aggregation effects of polymers in thin film will make the energy band gap of polymers narrower than in solution and the wavelength will shift toward red. The variation of energy band gap in polymers is not only due to the variation of molecular distances but also due to the polarity of solvents. FIG. 11 shows the UV-Vis absorption peak values for P1 and P2 dissolved in various solvents with different polarity. The peak absorption wavelengths did not increase with increasing polarity. It shifted to red first and then to blue because the highly polar solvent produced the orientation polarization effect and generated an electrical field with emission chromophores. The electrical field changes the basic state of the molecules.

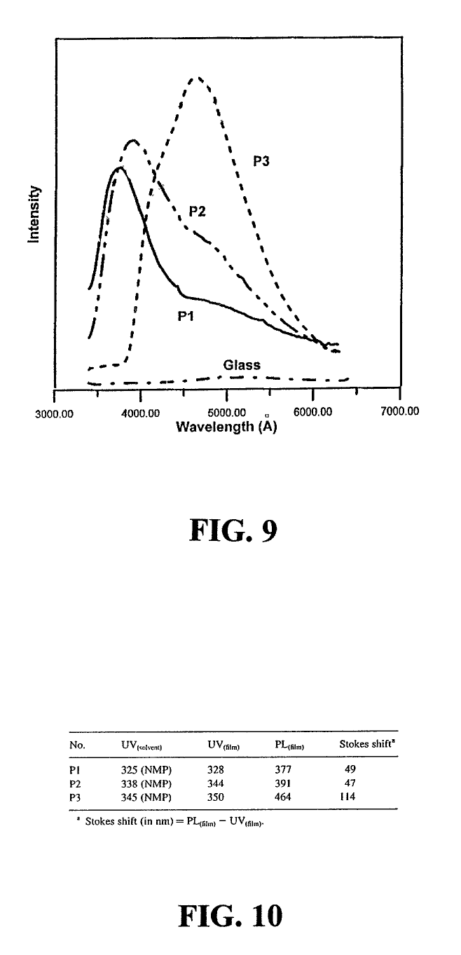

The PL spectra of the polymers are shown in FIG. 9. The luminescent peak wavelengths are shifted to longer wavelengths with increasing conjugating rings. The peak wavelengths are shown in FIG. 10. Although both P2 (with biphenyl) and P3 (with naphthalene) have two conjugating rings, the PL peak of P2 is shorter than P3 by 73 nm. It is due to the existence of a stereo torque angle between two benzene rings that breaks the conjugation plane and shortens the conjugation length. The luminescent wavelength of P2 is similar to P1 in red wavelength but shifted by 14 nm. The peak wavelength of P3 with naphthalene rings shifted 87 nm toward red relative to P1. It is seen that the conjugating effect from the biphenyl is much less than that from the naphthalene. All the polymers have pure luminescent spectra without other shoulder peaks due to the possession of only one kind of emission chromophore, therefore, the degree of polymerization had no effect on the width of the luminescent spectra. In FIG. 10, the Stokes shift is the difference between PL and UV-Vis absorption peaks. If the Stoke shift is too small, the emission and absorption spectra will overlap more. Then the emitting light will be self-absorbed and the luminescent efficiency will decrease in the devices.

1. A kind of synthesis and luminescent characteristic of novel phosphorus containing light-emitting polymers introducing eletro-transporting chromophores into an emission polymer to increase its electron affinity. Several phosphorus-containing emission chromophores are synthesized and incorporated with electron-transporting chromophores resulting in the novel phosphorus emitting blue light and improving thermal stability of resulting polymers such that the absorption peaks of these polymers are restricted to a stable range. The main characteristics are as follows:
First step: Before use, all reagents and solvents are reagent grade or purified by standard methods and each spectrum, melting points, nuclear magnetic resonance and optical characteristics are analyzed elementally.
Second step: 2,5-bis(4-fluorobenzoic acid)-1,3,4-oxadiazole is synthesized; to a reaction vessel is added (N₂H₄.H₂SO₄) 5 mmol and (4-fluorophenyl) 10 mmol, and 30 g polyphosphoric acid (PPA) solvent. The mixture is first heated to 150° C. for 8 h. to dissolve the reactants and then the reaction mixture is further heated at cyclization temperature (200° C.) for another 2 h. After completion of the reaction, the mixture is poured into deionized water to a white color pricipitate. The pricipitate is recrystallized from ethanol and dried to obtain a transparent gray crystal. Yield 73.9%; m.p 203˜204° C. This synthesis procedure is shown in FIG. 1.
Third step: synthesis of DOPO[2-(6-oxido-6H-dibenz<c,e><1,2>Oxaphosphorin-6-yl)] derivatives and [DOPO-BQ(p-benzoquinone), DOPO-BPQ(phenyl-p-benzoquinone), DOPO-NBQ(1,4-naphthoquinone)]. Derivatives of DOPO are synthesized according to FIG. 2 and are further recrystallized from ethoxyethanol.
Fourth step: syntesis of organic light-emitting polymer containing phosphorus. 1 mmol of 2,5-Bis(4-fluorophenyl)-1,3,4-oxadiazole, 1 mmol DOPO derivatives and 2.1 mmol K₂CO₃, 10 ml toluene and 5 ml NMP/CHP(N-methyl-2-pyrrolidone/N-cyclohexyl-2-pyrrolidone vol. ratio 1:1). The mixture is heated to 150° C. for 2 h under nitrogen and toluene is collected by a Dean-Stark trap. Then excess xylene is removed and the reaction mixture is heated at reflux (180° C.) for 20 h. After reaction, the mixture is poured into 300 ml acetone/methanol (vol. ratio 1:1), diluted with addition to 5 ml NMP and further stirred overnight. The precipitate is further filtered, washed with distilled water, extracted with chloroform for 24 h and dried under vacuum. The products are thus finally obtained.
